(19) United States
(12) Patent Application Publication
(10) Pub. No.: US 2026/0248660 A1
(43) Pub. Date: Aug. 27, 2026

(54) OSTOMY OUTPUT INDICATOR

(71) Applicant: Hollister Incorporated, Libertyville, IL (US)

(72) Inventors: Julie Gawenda, Chicago, IL (US); Jennifer Meza, Shorewood, IL (US); Rene Sieracki, Elmhurst, IL (US); Mary Ashley Tenedor, Plainfield, IL (US); Waeezah Waqar, Wheeling, IL (US); Abram Janis, Grayslake, IL (US)

(73) Assignee: Hollister Incorporated, Libertyville, IL (US)

(21) Appl. No.: 19/161,870

(22) PCT Filed: Mar. 15, 2024
(86) PCT No.: PCT/US2024/020231

§ 371 Date: Sep. 3, 2025

Related U.S. Application Data
(60) Provisional application No. 63/490,420, filed on Mar. 15, 2023.

(51) Int. Cl. A61F 13/42; A61F 13/84

(52) U.S. Cl. CPC A61F 13/42; A61F 2013/422; A61F 2013/8473

(57) ABSTRACT

A device for quantifying dejecta enzymes includes a plurality of layers, each layer including a hydrophobic material and a paper or paper-based material, the hydrophobic material being sealed to the paper or paper-based material. One of the plurality of layers is a first layer. The other layers define an assay column and a control column, the columns being isolated from one another by the hydrophobic material. In the assay column, the paper or paper-based material in a second layer includes an inhibitor or a dye or no inhibitor or dye. The paper or paper-based material in a third layer includes a colorimetric substrate, and the paper or paper-based material in a fourth layer includes no inhibitor or dye or colorimetric substrate. In the control column, the paper or paper-based material in the second layer includes no inhibitor or dye or colorimetric substrate, the paper or paper-based material in the third layer includes a dye, and the paper or paper-based material in a fourth layer includes no inhibitor or dye or colorimetric substrate. A method for quantifying dejecta enzymes is also disclosed.

### BACKGROUND

The present disclosure relates to an ostomy output indicator. More particularly, the present disclosure relates to a high throughput, rapid characterization indicator for dejecta enzymes

An ostomy is a procedure in which the ileum is brought outside of the body through a hole in the skin and abdominal wall called a stoma. The ileum is the point at which the small intestine ends, and is followed anatomically by the large intestine, or colon. The ostomy procedure can be performed to permanently or temporarily divert feces away from the colon.

During the procedure, a surgeon creates an incision into the abdomen and through the abdominal wall to reach the inside of the abdominal cavity. The resected part of the ileum is rerouted through another opening in the abdomen, forming the stoma. This procedure is most often prescribed due to conditions such as inflammatory bowel diseases, complicated diverticulitis, colon cancer, and sustained trauma to the colon.

In a permanent ostomy, the entire colon is removed whereas a temporary ostomy may be performed so that portions of the small or large intestine can heal. Afterward, the resected stoma can be reconnected to the intestine. An ostomy bag or pouch is a small pouch that collects diverted waste as it exits the stoma and serves as the new waste output regime for patients who have undergone an ostomy procedure. The ostomy bag is adhered to the skin surrounding the stoma, called peristomal skin.

Several ostomy and peristomal skin complications are known to occur. Such complications can include prolapse and stenosis, parastomal hernia, high output fistula, necrosis, and peristomal skin excoriation. Stoma prolapse occurs when part of the intestine is pushed out through the stoma, while stoma stenosis is when the stoma closes and does not allow for the passage of dejecta. A high output stoma is when there is an abnormally large output of dejecta, causing dehydration. Necrosis of the stoma can also occur in which the tissue begins to die and rejects back into the abdomen. Peristomal skin excoriation can also occur when the skin around the stoma. i.e., the peristomal skin, becomes stripped and damaged. This can lead to peristomal skin complications (PSCs), which are the most common of ostomy complications.

One study completed by Murken et al. found PSCs to have an incidence of 43% in one ileostomy patient population, but recent research suggests the actual percentage of affected ileostomates can reach up to 75%. This is detrimental to the physical and mental well-being of ostomy patients as PSCs have a wide range of negative effects. First, the damaged skin affects the adhesive seal of the ostomy pouch, which causes leakage of dejecta. This can lead the patient to experience pain, odor coming from the stoma site, irritation of the skin, and itching. Additionally, these complications lead to lengthier recoveries and higher cost of treatment.

Current research indicates that PSCs are the result of several physical and chemical factors at the site of skin around the stoma. For example, the adhesive which attaches the ostomy pouch to the peristomal skin is very strong. And, the removal of the pouch can cause stripping of the skin, which is worsened by other chemical interactions due to leakage of the dejecta. The liquid or solid waste matter excreted through the stoma into the ostomy bag, also known as dejecta, is approximately neutral and relatively alkaline compared to the acidic skin barrier (pH 4-5) and is enzyme rich. The pH imbalance results in irritation, while the highly enzymatic dejecta adds to the breakdown of the protein- and fat-rich skin. Additionally, the ostomy pouch must be changed when it becomes full, which can occur every few days or more often. Therefore, the cycle of skin stripping and skin breakdown allows the peristomal skin very little time to heal. Research has attempted to characterize this detrimental effect of ostomy dejecta on peristomal skin, as the exact physio-chemical mechanism of breakdown is not entirely understood.

Dejecta is highly enzymatic due to protein synthesis in the pancreas. Cells of the pancreas produce proteins at the highest rate of any human organ, and most of these proteins are digestive enzymes. Three main classes of digestive enzymes present in dejecta include: proteases; lipases; and amylases. Proteases cleave proteins, lipases cleave fats, and amylases cleave carbohydrates such as sugars. The pancreas secretes these enzymes into the intestinal lumen, where they assist in the breakdown of waste. However, the ostomy procedure diverts these enzymes in the waste out of the stoma while many of the enzymes are still active. The main components of skin are protein and lipids, and as such, proteases and lipases present in the dejecta are of particular importance for investigation. The protective barrier of the skin is held intact partly through the action of proteins. Thus, it is believed that proteases reduce the integrity of peristomal skin by cleaving these proteins. Similarly, the lipids in the skin can be broken down by lipases in the dejecta.

It is known that there is an associated link between PSCs and their emergence due to ostomy dejecta. However, the quantification of dejecta enzymes is not a standard practice, and current methods of enzyme detection or characterization of enzymatic activity include the enzyme-linked immunosorbent assay (ELISA) test and paper-based microfluidic devices known as microPADs (μPADs).

The enzyme-linked immunosorbent assay (ELISA) test is the gold standard method in applications ranging from food safety, environmental monitoring, and clinical diagnosis. There are many variations of the standard ELISA including the sandwich and competitive strategies. The ELISA test is typically performed for the detection and quantification of a target protein or macromolecule within an experimental sample. The general procedure for this technique is based on hyper-specific antigen-antibody recognition. It is combined with enzyme catalysis that allows for high efficiency of this method.

In one form of ELISA, a 96-well plate is utilized to bind the antibody-target macromolecule complex and undergoes several incubation and washing steps to ensure unbound materials are removed. After this, the plate is inserted in a microplate reader which allows for detection of the sample macromolecule at the bottom of the microplate via color intensity. The intensity of the color can then be measured, and a calibration curve can be created which plots the concentration of the given target protein or molecules as a function of color intensity. In this way, samples with unknown concentrations of a given protein or molecule of interest can be identified based on where they fall on the calibration curve and its respective color intensity.

Even though the procedure can be quite complex, due to its high-throughput nature and scalability, ELISA has been considered the best method for macromolecule detection. However, factors such as narrow thermophilic scope, short shelf life and easy inactivation or autophagy of enzymes can lend to the disadvantages of ELISA. Perhaps the most notable drawback is that ELISA can take up to 4 hours for completion and the high cost for ELISA materials, equipment, and facilities required to perform such procedures. In addition, proteases are proteins that can break down other proteases, so it would be expected that the sample integrity could be affected during the assay.

Another current method in development for enzyme quantification is based on μPADs. These devices are formed from paper patterned with hydrophobic barriers, where hydrophobic molecules are embedded within the fibers of chromatography paper using various methods. This process creates zones which guide a sample through the device via capillary action, as paper is inherently hydrophilic in nature. An advantage of μPAD devices in point-of-care (POC) applications are the low cost of paper, speed of assay, and ease of manufacture.

The small size also delivers portability, which the ELISA test lacks. One μpad device method for quantifying enzymes produced enzyme concentration results for alkaline phosphatase in a sample in 30 minutes. However, the design of the device incorporates specific substrates which require change depending on the target analyte. Some of these substrates are commercially available, while some are produced through chemical reactions which require an advanced understanding of biochemistry.

Colorimetric substrates, on the other hand, are a common type of substrate used in paper-based microfluidic devices. These materials can also be integrated as a detection method with photo or color sensing technology, which are readily available. Methods for producing the hydrophobic barriers in μPAD are also becoming more readily available. For example, one prior known method printed hydrophobic ink onto chromatography paper using a known document printer. One newer but costly method of producing hydrophobic barriers entails printing the wax layer using a fused deposition modeling (FDM) 3D printer and melting the wax into chromatography paper using an oven.

All of the known devices and methods have similar deficiencies: there is a lack of systems and methods for a high throughput, rapid characterization of ostomy dejecta enzymes for incorporation into products for mitigation of peristomal skin complications.

Accordingly, there is a need for an improved ostomy output indicator. Desirably, such an indicator provides rapid responses, is efficient and portable, and provides specificity to ostomy dejecta enzymes.

### BRIEF SUMMARY

In one aspect an improved ostomy output indicator device rapidly, and efficiently quantifies dejecta enzymes. The indicator device is portable and uses a paper-based microfluidic structure that can achieve sample detection based on colorimetric sensors.

In an embodiment, the indicator device includes a plurality of layers, each layer including a hydrophobic material and a paper or paper-based material. The hydrophobic material is sealed to the paper or paper-based material. One of the plurality of layers is a first layer, and the other layers define an assay column and a control column. The assay column and the control column are isolated from one another by the hydrophobic material.

In embodiments, in the assay column, the paper or paper-based material in a first of the other of the plurality of layers (e.g., the second layer) includes an inhibitor. In testing, a dye was used to change the color of the indicator for ease of visibility. The paper or paper-based material in a second of the other of the plurality of layers (e.g., the third layer) includes a colorimetric substrate, and the paper or paper-based material in a third of the other of the plurality of layers (e.g., the fourth layer) includes no inhibitor or dye or colorimetric substrate.

In embodiments, the, the inhibitor(s) can include but are not limited to the trypsin inhibitor 1-chloro-3-tosylamido-7-amino-2-heptanone (TLCK) to inhibit trypsin activity, the chymotrypsin inhibitor L-(tosylamido-2-phenyl) ethyl chloromethyl ketone (TPCK) to inhibit chymotryptic activity, or the elastase inhibitor Elastase Inhibitor I or 2, or combinations of these inhibitors. By inhibiting specific pancreatic proteases, it is possible to use a common colorimetric substrate and identify specific protease contributions to the proteolytic activity of the dejecta.

In embodiments, in the control column, the paper or paper-based material in the first of the other of the plurality of layers (e.g., the second layer) includes no inhibitor or dye or colorimetric substrate, the paper or paper-based material in a second of the other of the plurality of layers (e.g., the third layer) includes a dye, and the paper or paper-based material in a third of the other of the plurality of layers (e.g., the fourth layer) includes no inhibitor or dye or colorimetric substrate.

In embodiments, the hydrophobic material is wax and the assay and control columns are formed by openings in the wax in the others of the plurality of layers (e.g., the second through the fourth layers). The buffer layer includes an opening. In embodiments, the buffer layer includes a single opening in which a sample is introduced that flows into the assay and control columns.

The openings in the wax in the assay column are aligned with one another and the openings in the wax in the control column are aligned with one another. The assay column and the control column are spaced from one another. The paper or paper-based material can be a chromatography paper.

In embodiments, each layer of the plurality of layers is adhered to each adjacent layer. The layers can be laminated to further seal the plurality of layers into a single layered device.

In embodiments, in the assay column, the paper or paper-based material in the first of the other of the plurality of layers includes an inhibitor. Alternatively, the paper or paper-based material in the first of the other of the plurality of layers can include a dye to change the final indication color to enhance visibility. Alternatively still, the paper or paper-based material in the first of the other of the plurality of layers includes no inhibitor or dye.

The device can be configured to detect the concentration of chymotrypsin in a sample of ostomy output or dejecta. In such an embodiment, the inhibitor can be a protease inhibitor for proteases present in the dejecta, such as trypsin. Other inhibitors are contemplated and will be recognized by those skilled in the art. The colorimetric substrate in the assay column can be a chymotrypsin colorimetric substrate defined by Suc-Gly-Gly-Phe-pNA. In embodiments, in the assay column, the dye is a blue dye. In the control column, the dye can be a green.

In an aspect, a method of quantifying dejecta enzymes includes introducing a sample of dejecta into a device having a plurality of layers, each layer including a hydrophobic material and a paper or paper-based material, the hydrophobic material being sealed to paper or paper-based material, one of the plurality of layers being a first layer, the other of the plurality of layers defining an assay column and a control column, the assay column and the control column being isolated from one another by the hydrophobic material, in which in the assay column, the paper or paper-based material in a first of the other of the plurality of layers includes an inhibitor or a dye or no inhibitor or dye, the paper or paper-based material in a second of the other of the plurality of layers includes a colorimetric substrate, and the paper or paper-based material in a third of the other of the plurality of layers includes no inhibitor or dye or colorimetric substrate, and in which in the control column, the paper or paper-based material in a first of the other of the plurality of layers includes no inhibitor or dye or colorimetric substrate, the paper or paper-based material in a second of the other of the plurality of layers includes a dye, and the paper or paper-based material in a third of the other of the plurality of layers includes no inhibitor or dye or colorimetric substrate.

The method further includes comparing a color of the dejecta migrating through the device and appearing in the paper or paper-based material in the third of the other of the plurality of layers with a known color key.

Such a method can include a device that is configured to detect the concentration of chymotrypsin in a sample of ostomy output or dejecta. In such a method, in the device, the inhibitor is a protease inhibitor. Further, in such a method, the colorimetric substrate can be a chymotrypsin colorimetric substrate defined by Suc-Gly-Gly-Phe-pNA.

Using inhibitors such as the trypsin inhibitor 1-chloro-3-tosylamido-7-amino-2-heptanone (TLCK) to inhibit trypsin activity, the chymotrypsin inhibitor L-(tosylamido-2-phenyl) ethyl chloromethyl ketone (TPCK) to inhibit chymotryptic activity, or the elastase inhibitor Elastase Inhibitor I or 2, or combinations of these inhibitors, a method can includes use of a device that is configured to detect the concentration of trypsin in a sample of ostomy dejecta. In such a method, in the device, the inhibitor is a chymotrypsin inhibitor. Further, in such a method, the colorimetric substrate can be a chymotrypsin colorimetric substrate defined by Suc-Gly-Gly-Phe-pNA, which can also be cleaved by trypsin.

In some methods, in the assay column, the dye is a blue dye. In some methods, in control column, the dye is a green.

Other aspects and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 illustrates an embodiment of a multi-layer ostomy output indicator device, the illustrated device being a four layer device;

FIG. 2 is an illustration of the wax/paper sandwich including a wax support or substrate and chromatography paper for each of the layers;

FIGS. 3A and 3B are photographs of examples of the buffer layer and the second through fourth layers;

FIGS. 4A and 4B are illustrations of the buffer layer and the second through fourth layers

FIG. 5 is a five-image color key showing the color intensities, in which, from left to right, are the lowest concentration of chymotrypsin (the lightest color intensity) to the highest concentration of chymotrypsin (the darkest or greatest color intensity);

FIG. 6 illustrates the wax layer after it has been milled to the proper thickness and with the proper patterning (as shown with the assay and control columns present in layers 2 through 4) on a platform of a CNC mill, and showing a sheet of chromatography paper that is slightly larger than the perimeter of the wax layer to account for spreading of the wax during melting;

FIG. 7 illustrates pre-deposition of the liquid reagents in the areas of the layers in the second and third layers of in the assay column and in the third layer of the control column;

FIG. 8 is a graphical representation of the Feclone viscosity vs. likelihood of output for ileostomates and colostomates; and

FIG. 9 illustrates an example of a housing for the ostomy output indicator.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated.

Presently disclosed embodiments are portable indicator devices that can be used to rapidly, and efficiently quantify dejecta enzymes from ostomy output. The indicator devices are paper-based microfluidic devices that can achieve sample detection based on colorimetric sensors.

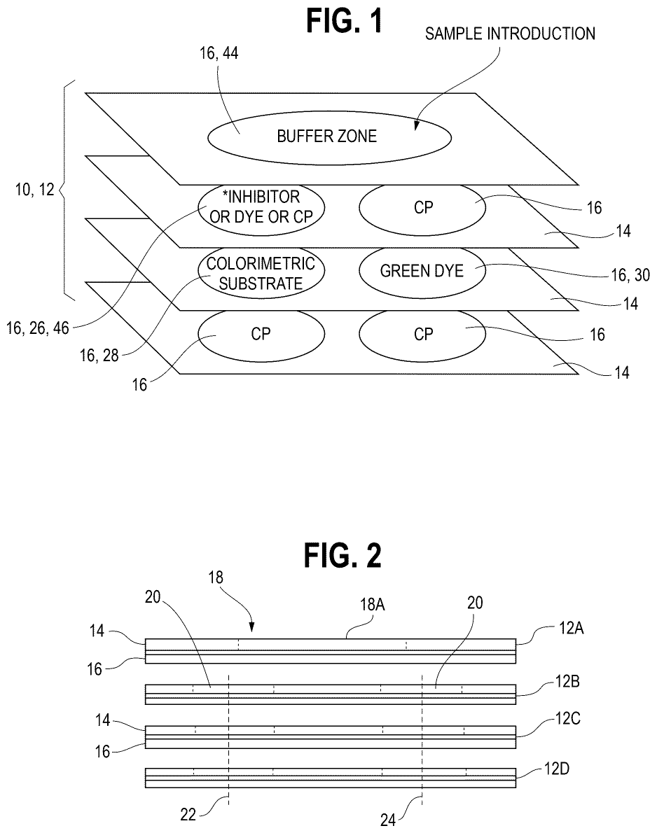

In an embodiment, the ostomy output indicator 10 is a multi-layer device that functions to detect the concentration of chymotrypsin in a sample of, for example, ostomy output or dejecta. In embodiments the indicator 10 has six layers, four layers, or more or less layers. A four layer embodiment is illustrated in FIG. 1.

Certain design criteria are laid out for the indicator device 10. Primary criteria include speed of results, portability (e.g., light weight and relatively small dimensions), the ability to be operated in a clinical setting, and ease of manufacture. The indicator device 10 should produce comparable results to current standards of immunoassays for the quantification of elastase, chymotrypsin, and trypsin enzymes.

Use of the indicator device 10 should rely mainly on color intensity detection and a larger viewing area for the user, and should produce results within a relatively short timeframe, for example, less than 60 minutes and preferably within 30 minutes. By using a color-changing substrate for detection, a wax/paper sandwich 12 can create a color indicator for chymotrypsin concentration, and do so in a meaningful timeframe.

Other design criteria include minimizing the sample (volume) required, buffering of the sample pH, and detection of chymotrypsin. For ostomy protease quantification the indicator device 10 desirably functions in a wide pH range (5-8) to conform to ostomy dejecta contents.

Referring to FIG. 2, the illustrated multi-layer design is formed as a wax/paper sandwich 12 and includes a wax support 14 or substrate and a paper or paper-based element 16 for each of the four layers 12a-12d. In embodiments, the paper or paper-based element 16 of the sandwich 12 is Whatman 1 filter paper, with pre-deposited liquid reagents facilitating a color intensity change depending on the concentration of chymotrypsin detected. Other types of suitable paper or paper-based material will be recognized by those skilled in the art. The multi-layered design illustrated in FIG. 1 includes four layers, one shared buffer introduction layer 12a and three chromatography paper layers 12b-12d. Each of the layers 12a-12d have a wax 14 base.

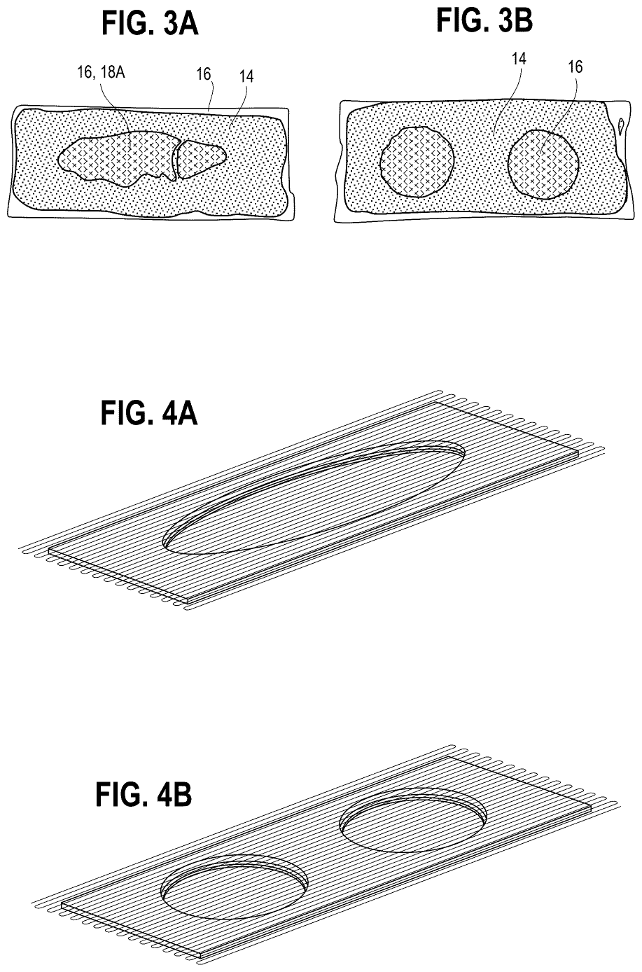

The first or buffer layer 12a includes a buffering zone 18 (see FIGS. 3A and 4A), which is a larger opening that permits access to the layers 12b-12d below. In the illustrated embodiment, the buffer layer 12a includes an elliptical opening 18a. Other shapes of openings will be appreciated from a study of the present disclosure. The three chromatography layers 12b-12d each include two circular regions 20 formed in the wax base 14 and in which the chromatography paper 16 is exposed. The circular regions 20 are formed in two parallel columns 22, 24 with the circular regions 20 in each column aligned with one another. In an embodiment, one column 22 is a test or assay column and the other column 24 is a control column. FIGS. 3A and 3B are photographs of examples of the buffer layer 12a and the second through fourth layers 12b-12d, and FIGS. 4A and 4B are illustrations of the buffer layer 12a and the second through fourth layers 12b-12d.

Each of the wax layers 14 includes appropriately sized holes, which can be made in fabrication. The buffer layer 12a includes the buffer zone 18, which is the larger opening 18a for introducing the dejecta or sample, as a sample may be a dilution of the dejecta or a supernatant of the centrifuged dejecta. Below the buffer layer 18 (i.e., the second layer 12b), the paper 16 in the assay column 22 includes a protease inhibitor 26 or dye 46 or untreated chromatography paper 16, and the control column 24 includes untreated chromatography paper 16. In the third layer 12c, the paper 16 in the assay column includes a colorimetric substrate 28 and the paper 16 in the control column 24 include chromatography paper 16 prepared with green dye to induce a color change. The final or fourth layer 12d includes chromatography paper 16 in both the assay and control columns 22, 24.

In fabrication, the openings 18a, 20 are formed in each wax layer 14. The openings 18a, 20 can be formed by milling into the wax substrate 14. Each wax layer 14 is melted onto (sealed on to) chromatography paper 16 and prepared accordingly, depending on whether it is a plain, untreated layer or a layer having a pre-deposited liquid reagent such as a dye 30,46, the colorimetric substrate 28, or an inhibitor 26. For those layers 12b, 12c having a pre-deposited liquid reagent, after melting, as described below, the liquid reagent is pre-deposited on the chromatography paper 16 of that layer.

The layers 12a-12d are subsequently properly positioned (layered) so that the open (chromatography paper 16 exposed) regions, forming the columns 22, 24 are aligned. The layers 12a-12d can be laminated to form an encapsulated device 10.

In the second layer 12b, in the assay column 22, an inhibitor, such as a trypsin inhibitor 26 can be pre-deposited to inhibit detection of trypsin by the colorimetric substrate 28, as the substrates for chymotrypsin and trypsin are similar. However, a dye 46, such as a blue dye, can be pre-deposited to alter the visible color of the chymotrypsin colorimetric substrate 28, which is yellow, in that changes in intensity may be difficult to detect. In the third layer 12c, in the assay column 22, the colorimetric substrate 28 can be pre-deposited and in the control column 24, a green dye, as indicated at 30, can be pre-deposited on the chromatography paper 16.

Samples (dejecta) will pass through the layers of chromatography paper 16 in a downward-flow motion. The rate of flow is controlled by the number of layers. In the control column 24, upon reaching the third layer 12c inducing color change, the sample will encounter green dye 30 and result in a green color change of the chromatography paper 16 (color change from white to green) emanating to the fourth layer 12d. In the assay column 22, upon reaching the third layer 12c inducing color change, the sample will encounter the colorimetric substrate 28. Depending on the concentration of the chymotrypsin within the sample, the colorimetric substrate 28 is cleaved by the chymotrypsin, producing the color change, and change to a color intensity reflecting the concentration. This will result in a color change of the chromatography paper 16 emanating to the fourth layer 12d. As noted above, a dye 46, such as a blue dye, may be pre-deposited on layer 12b in the assay column 22 to effect a visible green color at the fourth layer 12d to facilitate identification of the color change.

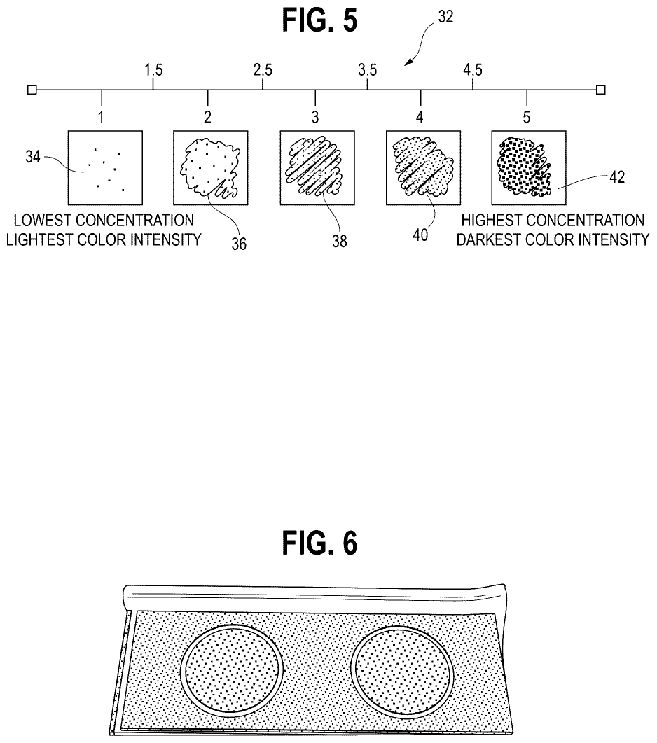

The present indicator device 10 can achieve sample detection based on visual analysis of protease quantification. Protease quantification is accomplished in two ways: intensity of color change and time difference of color detection between the assay and control columns 22, 24. The intensity of color change due to the colorimetric substrate 28, which bonds to the protease of interest, is a direct indicator of the concentration. It is believed that based on the concentration of protease, there may be a time difference of color detection between the assay and control columns 22, 24, i.e., if there is higher concentration of protease, it may result in a shorter time difference between color detected in the fourth layer 12d of the assay and control columns 22, 24. Prior to testing samples of dejecta with unknown concentrations of chymotrypsin, known amounts of chymotrypsin were tested in the present indicator device 10, and the intensity of the color as well as the time difference between color appearance in the assay and control columns 22, 24 was recorded and imaged to form a five-image color key 32, as illustrated in FIG. 5.

FIG. 5 illustrates the five-image color key 32, in which 1 (indicated at 34) corresponds to the lowest concentration of chymotrypsin (0 mg) and the lightest color intensity. Each increasing number (indicated at 2 (36) to 5 (42) has an increasing chymotrypsin concentration and darker color intensity. The concentration or quantity (in milligrams—mg) of chymotrypsin used to create the scale were: 1 (34)—0 mg; 2 (36)—50 mg; 3 (38)—100 mg; 4 (40)—150 mg; and 5 (42)—200 mg.

Unknown amounts of a protease could then be compared to this key 32 based on their color intensity to establish an approximate concentration of chymotrypsin within the sample.

In use of the indicator device 10, a user deposits a volume of the supernatant from a centrifuged sample in the sample introduction area 18a of the first layer 12a in a sterile environment. After about fifteen minutes, the user flips the wax/paper sandwich 14 and observes two colors on the fourth layer 12d, yellow in the assay column 22 and green in control column 24. The color in the assay column 22 is compared to the five-image color key 32, which will be similar to the depiction in FIG. 5, to determine which image is closest to the color visually observed. The concentration of chymotrypsin in the sample is determined from this comparison.

In fabrication of the indicator device 10, wax layers 14 were chosen as a hydrophobic barrier. Fabricating the wax layers 16 was through subtractive manufacturing. Stock pieces of wax, about ⅛″ thick were prepared and smoothed. Various iterations of wax dimensions were prepared and tested. The tested dimensions are shown Table 1, below. The third column below illustrates the buffer layer ellipse 18a dimensions. The fourth column below illustrates the dimensions for the lower layers 12b-12d, which are those that include the assay and control columns 22, 24. It will be noted that the fourth column indicates one embodiment in which six layers were used. It will be appreciated that the number of layers was varied in testing to determine the efficacy of the indicator device 10 with differing layers.

TABLE 1
Wax Layer Design Iterations

| | | Layer 1 Ellipse | Layers 2-6 Circle | |
|---|---|---|---|---|
| Iteration | Rectangular size Dimensions | Dimensions | Dimensions | |
| 1 | 20.0 mm × 10.0 mm × 0.4 mm | N/A | N/A | |
| 2 | 85.0 mm × 30.0 mm × 0.8 mm | 31.51 mm × 11.0 mm | 24.0 | mm |
| 3 | 40.0 mm × 15.0 mm × 1.0 mm | 15.0 × 3.0 mm | 4.0 | mm |
| 4 | 440.0 mm × 15.0 mm × 1.0 mm | 16.0 × 3.5 mm | 8.0 | mm |
| 5 | 85 mm × 30 mm × 1.0 mm | 31.51 mm × 11.0 mm | 24.0 | mm |

Referring again to FIG. 2, each layer 12a-12d of the wax paper sandwich is formed individually. In an embodiment, Whatman Chromatography Paper No. 1 was used as the paper 16 for each layer. The wax 14 was melted onto the chromatography paper 16 to ensure even heat distribution and melting of the wax. In this way, the hydrophilic channels (the sample introduction area 18a of the first layer 12a, and the assay and control columns 22, 24 in the second through fourth layer 12b-12d) are formed in the wax paper sandwich 12 in individual layers. That is, each layer 12a-12d is fabricated separately, and the colorimetric substrate 28, the buffer 44, the inhibitor or dye 26, 30 are pipetted into the exposed paper openings 16 of each layer. The layers 12a-12d are then adhered in the correct order atop one another and flattened together. Finally, the layers can be laminated with only the sample introduction zone 18a left uncovered by the laminate for sample introduction.

In an embodiment, the indicator device 10 includes three two-hole wax layers 12b-12d, and one one-hole buffer layer 12a at the top, as shown in FIG. 1. For each layer, as illustrated in FIG. 6, the chosen wax layer 14 (one-hole or two-hole) is placed on a sheet of chromatography paper 16 that is slightly larger than the perimeter of the wax layer 14 to account for spreading of the wax during melting.

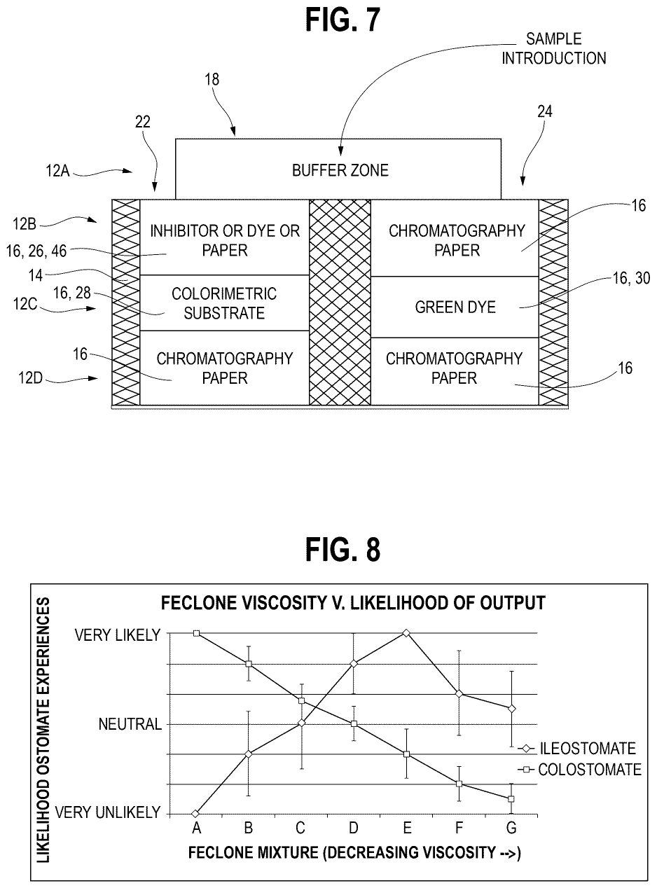

Following melting of the wax layers 14 onto the filter paper 16 (sealing the wax onto the filer paper), the liquid reagents 26, 28, 30, 44, as appropriate, are pre-deposited in the areas of the layers as shown in FIG. 7. Pre-deposition is carried out in the second and third layers 12b, 12c in the assay column 22 and in the third layer 12c in the control column 24. It is contemplated that the buffering liquid reagent 44 may be pre-deposited in the buffer layer 12a, however in a present embodiment, the buffer zone 18 is treated as a shared sample introduction region. In the assay column 22, the second layer 12b is utilized an area of experimentation, where either the inhibitor 26 or the dye 46 will be pre-deposited. The trypsin inhibitor 26 can be pre-deposited to inhibit detection of trypsin by the colorimetric substrate 28, as the substrates for chymotrypsin and trypsin are highly similar. Alternatively, it is envisioned that a dye 46 (for example, a blue dye) may be pre-deposited in the second layer 12b of the assay column 22 to alter the visible color of the chymotrypsin colorimetric substrate 28, which is yellow, because changes in color intensity may be difficult to detect. The third layer 12c in the assay 22 column is pre-deposited with the chymotrypsin colorimetric substrate 28 (Suc-Gly-Gly-Phe-pNA) while the third layer 12c in the control column 24 is pre-deposited with a commercially available green dye 30.

To determine the volumes of the pre-deposited materials, testing has equated pre-deposited volumes to the volume of the sample, which is assumed to be 25 μL per desired layer penetration depth. As such, 50 μL of the colorimetric substrate 28 and green dye 30 are respectively pre-deposited, to react with a 100 μL of sample introduced in the first layer 12a. The sample will pass through the layers of chromatography paper 16 in a downward-flow motion. The four layers control the rate of flow. In the control column 24, upon reaching the third layer 12c inducing color change, the sample will encounter the green dye 30, resulting in a green color change of the chromatography paper 16 (white to green) emanating to the fourth layer 12d. In the assay column 22, upon reaching the third layer 12c inducing color change, the sample will encounter the colorimetric substrate 28. Depending on the concentration of the chymotrypsin in the sample, the colorimetric substrate 28 will be cleaved by the chymotrypsin and change to a color intensity reflecting the concentration. This will result in a color change of the chromatography paper 16 emanating to the fourth layer 12d. FIG. 7 illustrates this mechanism in two columns, clearly showing the hydrophobic wax layers 14 which promote sample guidance downwards.

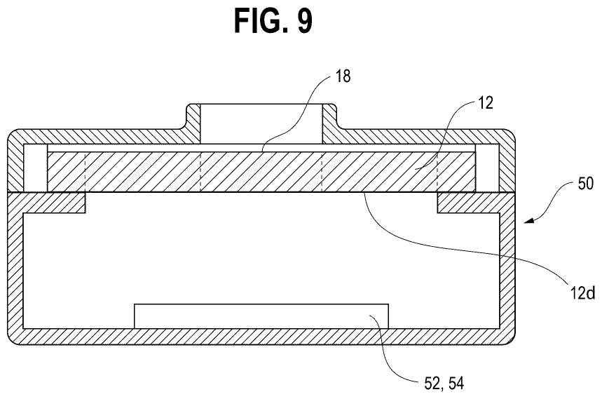

Referring to FIG. 9, in embodiments, the indicator device 10 can be enclosed in a housing 50 and can include a color sensor or color sensors 52, connected to a controller/microcontroller 54, such as an Arduino™ device. The housing 50 can enclose the sensor 52 and support the wax/paper sandwich 14. The color sensor 52 detects induced color changes in the last (bottom) layer 12d and can provide indication, for example, red-green-blue (RGB) values. The RGB values can be output to a display (not shown), such as an LCD display (not shown). Depending on the concentration of the protease within the sample, the intensity of the color and the difference in transit time of the sample through the assay and control columns 22, 24, enable two-fold quantification of concentration.

In other embodiments and use thereof, the indicator device 10 can rely on a visual analysis and comparison to a five-image ileometer color key 32. Samples of a material having known amounts of chymotrypsin were tested in the indicator device 10, and the intensity of the color, as well as the time difference between color appearance in the assay and control columns 22, 24 were recorded and imaged to form the five-image color key 32. Unknown amounts of a protease in dejecta can then be compared to the key 32 based on their color intensity to establish an approximate concentration of chymotrypsin in the sample.

In still other embodiments, a camera (not shown) can be positioned below the assay column 22 to capture images of color intensity. Using the images, MATLAB or ImageJ software can be used to derive color intensity values and plot them on a calibration curve.

To prepare the five-image ileometer color key 32, four samples of varying concentration of chymotrypsin (and one sample with no chymotrypsin) were prepared. The concentrations include 0% chymotrypsin, 50 mg of chymotrypsin in 1000 μL of 0.001 N HCl, 100 mg of chymotrypsin in 1000 μL of 0.001 N HCl, 150 mg of chymotrypsin in 1000 μL of 0.001 N HCl, and 200 mg of chymotrypsin in 1000 μL of 0.001 N HCl. The samples were prepared by reconstitution of chymotrypsin, Alpha, TLCK (bovine pancreas) material supplied as a dialyzed, lyophilized powder in 1000 μL of 0.001 N HCl.

The colorimetric substrate 28 was reconstituted with 5 μm of chymotrypsin colorimetric substrate (Suc-Gly-Gly-Phe-pNA) in 250 μL of Dimethyl sulfoxide (DMSO) stock solution. It was observed that the reaction of alpha-chymotrypsin from bovine pancreas with chymotrypsin colorimetric substrate (Suc-Gly-Gly-Phe-pNA) induced a faint yellow color on the chromatography papers which was not very clearly detectable in pictures. As such, the pre-deposited blue dye layer was incorporated in the wax-paper sandwich at layer 12b in the assay column 22 which made the final color appearance a more visible shade of green that was easily observable in the captured images and could therefore be used for image analysis.

In confirming/testing for adequate movement of dejecta sample and reagent flow laterally and vertically through the assay and control columns 22,24, i.e., that proper consistency for testing was present, it was found that sample preparation was required and the dejecta required centrifuging and extraction of the supernatant.

Artificial dejecta was simulated using Feclone, a material commonly used as simulated fecal material for industrial testing of diapers and ostomy bags. Samples were prepared of a Feclone solution centrifuged to extract the supernatant. This ratio was determined to be representative of ileostomate users. A range of mixtures were created and tested representing different dejecta consistencies with varying degrees of viscosity based on the likelihood of patients seeing these consistencies in real use. The ratio of mixtures was shown to be highly likely in ileostomate users. FIG. 8 illustrates the Feclone viscosity vs. likelihood of output for ileostomates and colostomates.

It will be appreciated that the present ostomy output indicator 10 provides users with a portable indicator device 10 that can be used to rapidly, and efficiently quantify dejecta enzymes from ostomy output. The indicator device 10 is a wax and paper-based sandwich that defines a microfluidic indicator that can achieve sample detection based on colorimetric sensors.

The indicator device 10 provides colorimetric results quickly in a portable device with the ability to be operated in a clinical setting. The indicator device 10 produces results comparable to current standards of immunoassays for the quantification of elastase, chymotrypsin, and trypsin enzymes. The device 10 provides results via color intensity detection within a relatively short timeframe. By using a color-changing colorimetric substrate 28 for detection, the device 10 creates a color indicator for determining chymotrypsin concentration, functioning in wide pH range (5-8) to conform to ostomy dejecta contents.

In the present disclosure the words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words such as, but not limited to, “upper” “lower” “raised” “lowered” “top” “bottom” “above” “below” “up” “down” “alongside” “front” “back” “left” and “right” and the like are used for purposes of illustration and as such, are not limiting. Depending on the context, the word “if” as used herein may be interpreted as “when” or “upon” or “in response to determining.”

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A device for quantifying dejecta enzymes comprising:
a plurality of layers, each layer including a hydrophobic material and a paper or paper-based material, the hydrophobic material being sealed to paper or paper-based material,
one of the plurality of layers being a first layer, the other of the plurality of layers defining an assay column and a control column, the assay column and the control column being isolated from one another by the hydrophobic material,
wherein in the assay column, the paper or paper-based material in a first of the other of the plurality of layers includes an inhibitor or a dye or no inhibitor or dye, the paper or paper-based material in a second of the other of the plurality of layers includes a colorimetric substrate, and the paper or paper-based material in a third of the other of the plurality of layers includes no inhibitor or dye or colorimetric substrate, and
wherein in the control column, the paper or paper-based material in a first of the other of the plurality of layers includes no inhibitor or dye or colorimetric substrate, the paper or paper-based material in a second of the other of the plurality of layers includes a dye, and the paper or paper-based material in a third of the other of the plurality of layers includes no inhibitor or dye or colorimetric substrate.

2. The device of claim 1, wherein the hydrophobic material is wax.

3. The device of claim 2, wherein the assay column and the control column are formed by openings in the wax in the others of the plurality of layers.

4. The device of claim 3, wherein the first layer is a buffer layer, the buffer layer includes an opening.

5. The device of claim 3, wherein the openings in the wax in the assay column are aligned with one another and the openings in the wax in the control column are aligned from one another, wherein the assay column and the control column are spaced from one another.

6. (canceled)

7. The device of claim 1, wherein in the assay column, the paper or paper-based material in the first of the other of the plurality of layers includes an inhibitor and/or a dye.

8. (canceled)

9. The device of claim 1, wherein in the assay column, the paper or paper-based material in the first of the other of the plurality of layers includes no inhibitor or dye.

10. The device of claim 1, wherein the paper or paper-based material is a chromatography paper.

11. The device of claim 1, wherein each layer of the plurality of layers is adhered to each adjacent layer.

12. (canceled)

13. The device of claim 1, wherein the device is configured to detect the concentration of chymotrypsin in a sample of ostomy output or dejecta.

14. The device of claim 13, wherein the inhibitor is a protease inhibitor.

15. The device of claim 1, wherein the inhibitor is one or more of 1-chloro-3-tosylamido-7-amino-2-heptanone (TLCK), L-(tosylamido-2-phenyl) ethyl chloromethyl ketone (TPCK), elastase inhibitor I or 2, and combinations thereof.

16. The device of claim 1, wherein the colorimetric substrate is a chymotrypsin colorimetric substrate defined by Suc-Gly-Gly-Phe-pNA.

17. The device of claim 1, wherein in the assay column, the dye is a blue dye, and the dye is a green dye in the control column.

18. (canceled)

19. A method of quantifying dejecta enzymes comprising:
introducing a sample of dejecta into a device having a plurality of layers, each layer including a hydrophobic material and a paper or paper-based material, the hydrophobic material being sealed to paper or paper-based material, one of the plurality of layers being a first layer, the other of the plurality of layers defining an assay column and a control column, the assay column and the control column being isolated from one another by the hydrophobic material,
wherein in the assay column, the paper or paper-based material in a first of the other of the plurality of layers includes an inhibitor or a dye or no inhibitor or dye, the paper or paper-based material in a second of the other of the plurality of layers includes a colorimetric substrate, and the paper or paper-based material in a third of the other of the plurality of layers includes no inhibitor or dye or colorimetric substrate, and
wherein in the control column, the paper or paper-based material in a first of the other of the plurality of layers includes no inhibitor or dye or colorimetric substrate, the paper or paper-based material in a second of the other of the plurality of layers includes a dye, and the paper or paper-based material in a third of the other of the plurality of layers includes no inhibitor or dye or colorimetric substrate; and
comparing a color of the dejecta migrating through the device and appearing in the paper or paper-based material in the third of the other of the plurality of layers with a known color key.

20. The method of claim 19, wherein the device is configured to detect the concentration of chymotrypsin in a sample of ostomy output or dejecta.

21. The method of claim 19, wherein the inhibitor is a protease inhibitor.

22. The method of claim 19, wherein the inhibitor is one or more of 1-chloro-3-tosylamido-7-amino-2-heptanone (TLCK), L-(tosylamido-2-phenyl) ethyl chloromethyl ketone (TPCK), elastase inhibitor I or 2, and combinations thereof.

23. The method of claim 19, wherein the colorimetric substrate is a chymotrypsin colorimetric substrate defined by Suc-Gly-Gly-Phe-pNA.

24. The method of claim 19, wherein in the assay column, the dye is a blue dye, and the dye is a green dye in the control column.

25. (canceled)
